(19) United States
(12) Patent Application Publication
(10) Pub. No.: US 2012/0282235 A1
(43) Pub. Date: Nov. 8, 2012

(54) IMPLANTABLE PATCH AND SURGICAL KIT FOR PREPARATION THEREOF

(76) Inventors: Kevin L. Ohashi, Jamaica Plain, MA (US); Dale R. Peterson, La Jolla, CA (US); Jamal Rushdy, Eden Prairie, MN (US)

(21) Appl. No.: 13/498,284

(22) PCT Filed: Jun. 1, 2010
(86) PCT No.: PCT/US10/36876

§ 371 Date: Jul. 6, 2012

Related U.S. Application Data
(60) Provisional application No. 61/246,016, filed on Sep. 25, 2009.

(51) Int. Cl. A61K 35/12; A61K 38/48; A61K 38/18; B01D 43/00; A61K 35/14; A61K 35/28; B65D 77/04; A61J 1/00; A61K 38/36; A61K 33/06

(52) U.S. Cl. 424/93.71; 424/93.7; 514/13.6; 424/94.64; 514/7.6; 424/682; 424/93.72; 424/520; 206/570; 206/205; 494/37

(57) ABSTRACT

Embodiments of the present invention include a surgical kit for preparation of a patch for implantation into a human body, characterized in that it comprises a package containing, on one hand, a synthetic substrate (1) and, on the other hand, means for treatment of the substrate with a solution (3) including at least one biologically active component (4, 5), adapted to integrate the biologically active component with the substrate. Embodiments of the invention include a patch for implantation into the human body, comprising a synthetic substrate (1), characterized in that at least one biologically active component (4, 5) is integrated with the substrate using the surgical kit.

### CROSS-REFERENCE TO RELATED APPLICATIONS

This application claims the benefit of U.S. Provisional Patent Application Ser. No. 61/246,016, filed on Sep. 25, 2009. The aforementioned application is incorporated by reference herein in its entirety for all purposes.

### TECHNICAL FIELD

The present invention is generally related to the field of tendon and ligament soft tissue repair. In particular, the present invention is related to a pre-packaged patch with platelet-enriched plasma and a kit for the preparation thereof.

### BACKGROUND

Removing tissue from one part of a patient's body for use on another part, such as, for example, a tissue repair, is often associated with increased surgical cost and time. Using synthetic patches for such ligament or tissue repair may often pose acceptance challenges and, in some cases, increased time required for healing or ingrowth. Constructing such patches from non-human tissue may involve increased time and expense, and may not give rise to the same strength or durability properties found in a synthetic patch.

### SUMMARY

In one embodiment, the present invention is a system with concentrated platelet-rich plasma and growth factors embedded within a synthetic patch. In another embodiment, the present invention is a patch system and kit. The patch system may be provided as a pre-packaged system including a synthetic patch positioned within a tube.

A surgical kit according to embodiments of the present invention includes a packaging containing a synthetic substrate and an instrument or tool for applying to the substrate or treating the substrate with a biologically active component, configured to integrate the biologically active component with the substrate. Such a surgical kit permits practical preparation of the implantable substrate, reducing cost and time necessary for such a surgical intervention.

Due to the integration of the biologically active component with the synthetic substrate, the body's acceptance of the substrate (e.g. the patch) is improved, according to embodiments of the present invention. Healing and ingrowth of soft tissue is facilitated, while reducing the cost and time necessary for the implantation of the surgical patch.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 illustrates a perspective view of a substrate, according to embodiments of the present invention.

FIG. 2 illustrates a perspective view of a tube and a fixation mechanism, according to embodiments of the present invention.

FIG. 3 illustrates a perspective view of the substrate of FIG. 1 positioned in the tube of FIG. 2, according to embodiments of the present invention.

FIG. 4 illustrates a perspective view of the addition of one or more biologically active components to the tube, according to embodiments of the present invention.

FIG. 5 illustrates a perspective view of multiple tubes placed into a centrifuge, according to embodiments of the present invention.

FIG. 6 illustrates a perspective view of a substrate enriched and/or coated with a biologically active component, according to embodiments of the present invention.

FIG. 7 illustrates a kit having a tube, a fixation mechanism, and a substrate, according to embodiments of the present invention.

FIG. 8 illustrates a kit having a tube and a substrate, according to embodiments of the present invention.

FIG. 9 illustrates a kit having a pipette and a substrate, according to embodiments of the present invention.

FIG. 10 illustrates a kit having a syringe and a substrate, according to embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

FIG. 1 illustrates a synthetic substrate 1 in the form of a disc, according to embodiments of the present invention. In one embodiment, the synthetic patch 1 is made of an absorbable polymer such as polyhydroxyalkanoate (PHA). An example of a commercially available synthetic patch is a TephaFlex® patch. The synthetic patch 1 may be a mesh patch or a woven patch, for example.

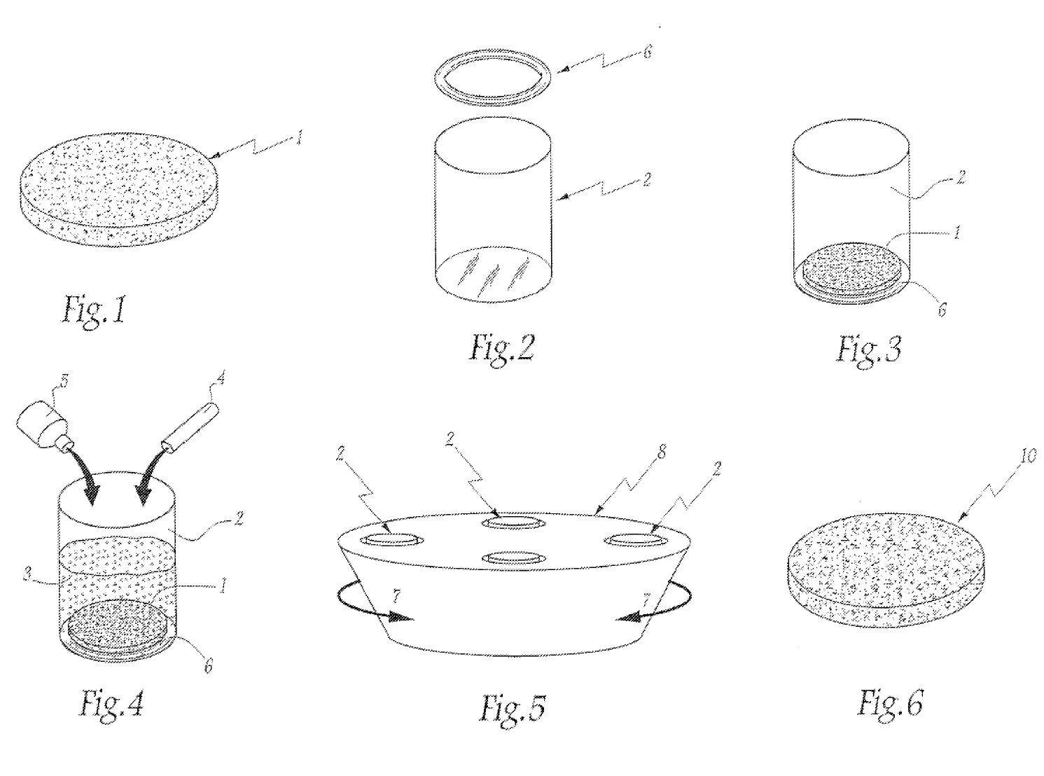

FIG. 2 illustrates a tube 2, which is empty before being filled by a liquid, according to embodiments of the present invention. The diameter of the tube 2 is substantially equal to the diameter of the synthetic substrate 1, according to embodiments of the present invention.

FIG. 3 illustrates the substrate 1 placed within an interior of the tube 2, positioned at one of the ends of the tube 2, according to embodiments of the present invention. In some cases, the substrate 1 may be held in a particular position in the tube 2 by a removable fixation mechanism 6, according to embodiments of the present invention. The position of synthetic patch 1 may optionally be maintained in the tube 2 by a fixation mechanism 6 such as, for example, a silicone o-ring, multiple o-rings, and/or by one or more lips or detents formed in the inside of the tube 2, according to embodiments of the present invention.

FIG. 4 illustrates the filling of the tube 2 with a solution 3, including at least one biologically active element 4, 5. The solution 3 may include different biologically active components 4, 5, including human blood and/or platelet-rich plasma 4, mixed with one or more other components 5.

As illustrated in FIG. 5, once the tube 2 is filled and/or partially filled with the solution 3, the tube 2 may be placed into a centrifuge device 8 for attaching the biologically active components 4, 5 to the substrate 1, according to embodiments of the present invention. Turning the centrifuge 8 in either of the directions indicated by arrows 7 separates the platelet rich plasma from the other blood products, according to embodiments of the present invention. For example, treating the solution 3 in centrifuge 8 causes the platelet rich plasma to sink toward the bottom of the tube 2, corresponding to a level within the tube 2 at which the synthetic substrate 1 is positioned. This causes the synthetic substrate 1 to absorb and/or otherwise attach to platelet rich plasma and/or the other components or agents separated out of the solution 3, according to embodiments of the present invention. According to embodiments of the present invention, component 4 is human blood or platelet rich plasma or fibrin, and component 5 is another agent, such as, for example, a binding agent.

FIG. 6 illustrates a patch ready for implantation, after treatment 7 and withdrawal from tube 2.

FIG. 7 illustrates a package 110 of the surgical kit 100, comprising an opening mechanism 111 and containing a synthetic substrate 1, a tube 2, and a fixation mechanism 6, according to embodiments of the present invention. Alternatively, the package 110 may include only a tube 2 and the fixation mechanism 6, according to embodiments of the present invention.

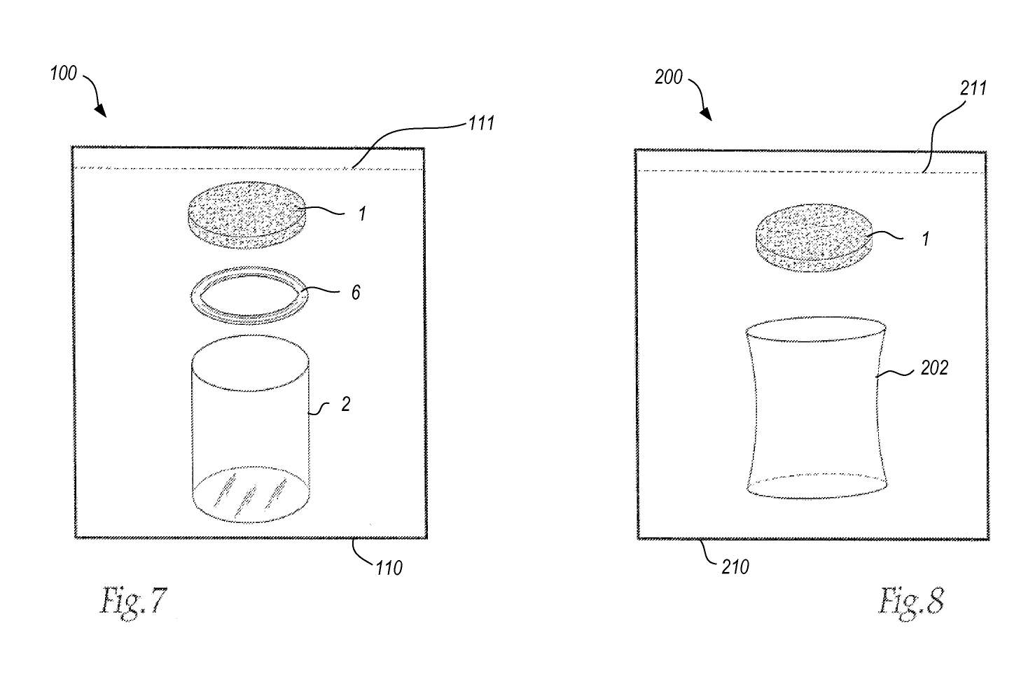

FIG. 8 illustrates a surgical kit 200 contained within a package 210 provided with an opening mechanism 211, a synthetic substrate 1, and a flexible pouch 202, according to embodiments of the present invention. The flexible pouch 202 may be used in a manner similar to tube 2 in order to provide an enclosure or partial enclosure in which solution 3 may be separated and/or mixed and/or coated onto synthetic substrate 1, according to embodiments of the present invention.

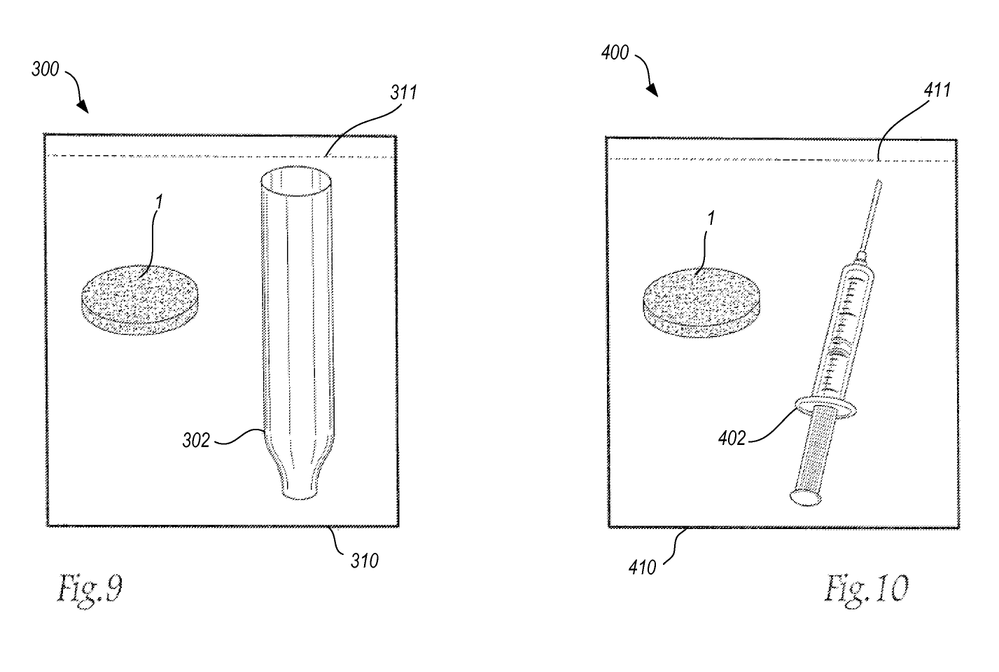

FIG. 9 illustrates a surgical kit 300 contained within a package 310 provided with an opening mechanism 311, a synthetic substrate 1, and a pipette 302, according to embodiments of the present invention. The pipette 302 may be used for placing the solution 3 onto the substrate 1.

FIG. 10 illustrates a surgical kit 400 contained within a package 410 provided with an opening mechanism, a synthetic substrate 1, and a syringe 402, according to embodiments of the present invention. Syringe 402 may be configured to inject the solution 3 into the substrate 1.

According to some embodiments of the present invention, kits 100, 200, 300, 400 may include a filter (not shown) for filtering solution 3. According to other alternative embodiments of the present invention, a surgical kit includes a synthetic substrate 1 with multiple treatment and/or deposition mechanisms as described above, or of another known type, configured to be contained within a package 410. As such, the surgical kit may be lightweight, inexpensive, and easy to use.

The contents of kits 100, 200, 300, 400, as well as their packages 110, 210, 310, 410, should remain as sterile as possible. Furthermore, the opening mechanisms 111, 211, 311 and 411 are configured to preserve such sterility, having, for example, a precut line for easy opening of the package, according to embodiments of the present invention.

One advantage of the platelet-rich plasma patch 10 is that the platelet-rich plasma 4 embedded into or onto the synthetic patch 1 can be from the blood of a patient with which the synthetic patch 10 will be used, and/or may be obtained from a blood bank. The platelet-rich plasma patch 10 may be used in soft tissue repair applications. For example, the platelet-rich plasma patch 10 may be used in tendon or ligament soft tissue repair.

According to some embodiments of the present invention, the synthetic patch 1 is mounted within the tube 2 at the appropriate level corresponding to the density of the platelet-rich plasma based on the volume of the blood that will be separated into plasma and the inner geometry of the tube 2. According to such embodiments, the blood 4 is inserted directly into the tube 2 and the tube 2 and patch 1 combination may be spun, such as, for example, by centrifuge. According to other embodiments of the present invention, the platelet-rich plasma (or other component) may be created at a separate time or location and then deposited or injected onto the patch 1, for example at a time just prior to or during surgery, as facilitated by kits 100, 200, 300, 400.

According to some embodiments of the present invention, platelet-rich plasma 4 is “spun down” (e.g. by centrifuge or the like) and/or injected onto the patch 4. According to other embodiments, one or more of the following elements is “spun down,” injected onto, and/or otherwise deposited onto the patch 1: platelet-rich plasma, platelet-poor plasma, bone marrow aspirate, cells such as platelets, white blood cells, stem cells (e.g. adipose or other types), and/or other biologic material.

Blood and other agents 5 may be added into the synthetic patch 1 by adding them into the tube 2. For example, other agents that may be added include, but are not limited to, growth factors for tissue growth and repair or protein coagulation. An example of a suitable protein coagulator includes, but is not limited to, thrombin. Another example of a suitable coagulator includes, but is not limited to, calcium ion. The platelet-rich plasma 4 is concentrated directly onto the synthetic patch 1, for example, by spinning the platelet-rich plasma 4 onto the synthetic patch 1. The blood and agents 5 may be concentrated into the synthetic patch 1 using, for example, a centrifuge 8 and/or filter. As the tube 2 is spun, the plasma is separated from other blood products and the platelet-rich plasma 4 is embedded onto the synthetic patch 1. According to some embodiments, components 4 and 5 have complementary effects.

The platelet-rich plasma patch 10 may be provided as a pre-packaged system and kit including a synthetic patch 1 and a tube 2, as illustrated in FIG. 7. In use, blood and other agents to be embedded onto the synthetic patch 1 are added into the tube 2. In one embodiment, the blood used to fill the tube 2 is the blood of the patient with which the synthetic patch 1 will be used. In this case, the platelet-rich plasma patch 10 may be formed during surgery. In other cases, the platelet-rich plasma patch 10 may be formed prior to surgery, or formed at another time. In one embodiment, the tube 2 may also be filled with a gelatinous material that may include agents 5 to be embedded into or onto the synthetic patch 1. After the blood and other agents 5 are added to the tube 2, the tube 2 is then spun to separate the platelet-rich plasma 4 from the blood and to concentrate the platelet-rich plasma 4 and other agents 5 into the synthetic patch 1. The platelet-rich plasma patch 10 can be used on a patient for tendon or ligament soft tissue repair, for example.

Instead of tube 2, numerous other variations of containers or partial containers may be used to hold the liquid or gelatinous solution 3, and a biomaterial compatible with one or more components 4, 5. According to embodiments of the present invention, the tube 2 or other container used may be of a form adapted to that of the substrate 1. For example, if the substrate 1 is square, the cross-sectional perimeter of the tube 2 may be square. In some cases, the external surface of the tube 2 or other container may be configured to fit within and/or interface with a compartment in the centrifuge 8, according to embodiments of the present invention. The internal surface of the tube 2 or other receptacle is adapted to receive and permit assembly of the substrate 1, according to embodiments of the present invention.

According to embodiments of the present invention, the optional fixation mechanism 6 is configured to position and/or hold the synthetic substrate 1 at one of the ends of the tube 2 or at any level in between. According to the position of the substrate 1 within the tube 2 or other receptacle, the volume and/or concentration of the solution 3 placed into the tube 2 can be varied, as well as the quantity and/or the concentration of the biologically active components 4, 5.

According to embodiments of the present invention, the plasma rich platelets may be separated from a blood or bone marrow sample. The separation and/or filtration may be accomplished without the use of a centrifuge, according to embodiments of the present invention. The components 4 and 5 resulting from the filtration may be transferred directly to the interior of the substrate 1 by changing from an aqueous phase to a non-aqueous phase, according to embodiments of the present invention.

According to embodiments of the present invention, the steps for preparing the patch may be repeated in order to add other components 4, 5, for instance just before or during surgery.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

1. A surgical kit (100; 200; 300; 400) for preparing a patch 10 for implantation into a human body, characterized in that it comprises a package (110; 210; 310; 410) containing, on one hand, a synthetic substrate (1) and, on the other hand, means for treatment (2, 6; 202; 302; 402) of the substrate with a solution (3) including at least one biologically active component (4, 5), adapted to combine the biologically active component with the substrate.

2. The surgical kit (100) according to claim 1, characterized in that the means for treatment comprise a receptacle (2), in particular a tube, adapted to receive the substrate (1) and to be filled with the solution (3).

3. The surgical kit (100) according to claim 2, characterized in that a lip is formed on the interior of the receptacle (2), the lip being adapted to receive and fixedly retain the substrate (1) in the receptacle (2).

4. The surgical kit (100) according to claim 2, characterized in that the means for treatment comprises a fixation mechanism (6), in particular a silicone ring, adapted to fixedly retain the substrate (1) in the receptacle (2).

5. The surgical kit (200) according to claim 1, characterized in that the means for treatment comprises a flexible pouch (202).

6. The surgical kit (300) according to claim 1, characterized in that the means for treatment comprises a pipette (302).

7. The surgical kit (400) according to claim 1, characterized in that the means for treatment comprises a syringe (402).

8. The surgical kit according to claim 1, characterized in that the synthetic substrate (1) is an absorbable polymer, in particular polyhydroxyalkanoate.

9. A patch (10) for implantation into a human body, comprising a synthetic substrate (1), characterized in that at least one biologically active component (4, 5) is integrated with the substrate using a surgical kit according to claim 1.

10. The patch according to claim 9, characterized in that each of the biologically active components (4,5) is one or more of the following: platelet enriched plasma (4), platelet poor plasma, bone marrow aspirate, platelet cells, white blood cells, stem cells, growth factors, protein coagulators, fibrin, prothrombin, thrombin, and calcium ions.

11. A method for preparing a synthetic substrate for implantation, the method comprising:
opening a sterile bag containing the synthetic substrate and a receptacle;
inserting the synthetic substrate into the receptacle;
filling the receptacle at least partially with human blood; and
subjecting the receptacle to centrifugal forces to separate plasma rich platelets from the blood and to concentrate the plasma rich platelets at a level within the receptacle corresponding to a location of the synthetic substrate.

12. The method of claim 11, further comprising implanting the synthetic substrate to repair human soft tissue.

13. The method of claim 12, wherein the human blood and the human soft tissue are from a single patient.

14. The method of claim 11, further comprising positioning the synthetic substrate at a selected level within the receptacle.

15. The method of claim 14, wherein positioning the synthetic substrate comprises inserting an o-ring into the receptacle below the synthetic substrate.

16. The method of claim 14, wherein positioning the synthetic substrate comprises inserting an o-ring into the receptacle above the synthetic substrate.

17. The method of claim 14, wherein an inner surface of the receptacle comprises at least one protrusion or indentation configured to facilitate positioning of the synthetic substrate within the receptacle.

18. A surgical kit for preparing a synthetic substrate for implantation, the surgical kit comprising:
a sealed bag, wherein an inside of the sealed bag is sterile;
a container within the sealed bag; and
a synthetic substrate within the sealed bag, wherein the synthetic substrate is biocompatible,
wherein the container is configured to hold a solution containing one or more biologically active components for combining the one or more biologically active components with the synthetic substrate.

19. The surgical kit of claim 18, wherein the container is a container selected from the group consisting of: a syringe, a pipette, and a flexible pouch.

20. The surgical kit of claim 18, wherein the synthetic substrate is positioned within the container within the sealed bag.

21. The surgical kit of claim 18, wherein the synthetic substrate is substantially circular, and wherein the container is a tube with a substantially circular inner diameter.

22. The surgical kit of claim 18, further comprising a protein coagulator.

23. The surgical kit of claim 22, wherein the protein coagulator is thrombin or calcium ion.

24. The surgical kit of claim 18, further comprising a growth factor.

25. The surgical kit of claim 18, wherein the synthetic substrate is an absorbable polymer.

26. The surgical kit of claim 25, wherein the absorbable polymer is polyhydroxyalkanoate.

27. The surgical kit of claim 18, further comprising a fixation mechanism, the fixation mechanism configured to hold the synthetic substrate at a desired position within the container.
